(19) United States
(12) Patent Application Publication
(10) Pub. No.: US 2018/0311236 A1
(43) Pub. Date: Nov. 1, 2018

(54) USE OF MASITINIB AND OTHER MAST CELL INHIBITORS FOR TREATMENT OF PARKINSON'S DISEASE

(71) Applicant: AB SCIENCE, Paris (FR)

(72) Inventors: Alain MOUSSY, Paris (FR); Jean-Pierre KINET, Lexington, MA (US); Colin MANSFIELD, Écully (FR)

(21) Appl. No.: 15/771,914

(22) PCT Filed: Oct. 28, 2016
(86) PCT No.: PCT/EP2016/076160

§ 371 Date: Apr. 27, 2018

(30) Foreign Application Priority Data
Oct. 28, 2015 (EP) 15306721.0

(51) Int. Cl. A61K 31/496; A61K 45/06; A61P 25/16

(52) U.S. Cl. CPC A61K 31/496; A61P 25/16; A61K 45/06

(57) ABSTRACT

Disclosed is a mast cell inhibitor, a pharmaceutical composition and a method for the treatment of patients afflicted with Parkinson's disease, wherein the patients are treated with a tyrosine kinase inhibitor or mast cell inhibitor, in particular masitinib, or a compound selected from imatinib, cromolyn sodium, midostaurin, BLU-285, bosutinib, ibrutinib, LAS189386, DP-2618, fostamatinib, dasatinib, sunitinib, axitinib, pazopanib, and toceranib or a pharmaceutically acceptable salt or solvate thereof, optionally in combination with at least one pharmaceutically active ingredient.

### FIELD OF INVENTION

The present invention relates to a mast cell inhibitor, a pharmaceutical composition and a method for treating patients afflicted with Parkinson's disease, wherein said patients are treated with a tyrosine kinase inhibitor or mast cell inhibitor, in particular masitinib or a pharmaceutically acceptable salt or solvate thereof, optionally in combination with at least one pharmaceutically active ingredient.

### BACKGROUND OF INVENTION

Parkinson's disease is a progressive degenerative disorder of the central nervous system characterized by insufficient formation and activity of dopamine produced in certain neurons.

Neuroinflammation is now established as an important aspect of pathology in Parkinson's disease. Data from post-mortem studies provided the first evidence for neuroinflammatory processes in 1988, with the reported presence of activated microglial cells within the substantia nigra of Parkinson's disease patients [McGeer PL, Itagaki S, Boyes BE, McGeer EG. Reactive microglia are positive for HLA-DR in the substantia nigra of Parkinson's and Alzheimer's disease brains. Neurology 1988; 38: 1285-91]. Studies of biological fluids (serum or cerebrospinal fluid) also support a role for neuroinflammatory processes in Parkinson's disease, revealing increased expression of interleukin 2, TNFα, and interleukin 6 [Stypula G, Kunert-Radek J, Stepieri H, Zyliriska K, Pawlikowski M. Evaluation of interleukins, ACTH, cortisol and prolactin concentrations in the blood of patients with Parkinson's disease. Neuroimmunomodulation 1996; 3: 131-34]; [Dobbs RJ, Charlett A, Purkiss AG, Dobbs SM, Weller C, Peterson DW. Association of circulating TNF-alpha and IL-6 with ageing and parkinsonism. Acta Neurol Scand 1999; 100: 34-41]. Proinflammatory changes have also been reported in the cerebrospinal fluid of patients with Parkinson's disease, with the detection of elevated TNFα, interleukin 1β, and interleukin 6 [Mogi M, Harada M, Riederer P, Narabayashi H, Fujita K, Nagatsu T. Tumor necrosis factor-alpha (TNF-alpha) increases both in the brain and in the cerebrospinal fluid from parkinsonian patients. Neurosci Lett 1994; 165: 208-10]; [ Blum-Degen D, Muller T, Kuhn W, Gerlach M, Przuntek H, Riederer P. Interleukin-1 beta and interleukin-6 are elevated in the cerebrospinal fluid of Alzheimer's and de novo Parkinson's disease patients. Neurosci Lett 1995; 202: 17-20]; [Muller T, Blum-Degen D, Przuntek H, Kuhn W. Interleukin-6 levels in cerebrospinal fluid inversely correlate to severity of Parkinson's disease. Acta Neurol Scand 1998; 98: 142-44]. These findings indicated that inflammatory changes are detectable during the course of Parkinson's disease before the death of the patients and are associated with progression of the disease. Such inflammatory cytokines are important mediators of harmful inflammation and among those considered of particular relevance to neuroinflammatory processes in Parkinson's disease are TNFα, interleukin 1β, and interferon γ [Hirsch EC, Hunot S., Neuroinflammation in Parkinson's disease: a target for neuroprotection? Lancet Neurol. 2009 Apr;8(4):382-97].

Nitric oxide (NO) is also known as having an important role in the pathogenesis of Parkinson's disease. The action of NO may have both positive and negative effects on the development of the disease, with one deleterious role being that NO and its progenitors are potentially toxic molecules and have been related to NO-mediated damage to neurons, which exacerbate the process of neurodegeneration. Post mortem analysis of Parkinson's disease brains have revealed higher than normal levels of NO producing enzyme, nitric oxide synthase (NOS) in the nigrostriatal regions [Hunot S, Boissiere F, Faucheux B, Brugg B, Mouatt-Prigent A, Agid Y, Hirsch EC, Nitric oxide synthase and neuronal vulnerability in Parkinson's disease, Neuroscience. 1996 May;72(2):355-63]. The involvement of NO in the pathogenesis of Parkinson's disease was also confirmed by various experimental models using neurotoxins [Kavya R, Dikshit M. Role of nitric oxide/nitric oxide synthase in Parkinson's disease. Annals of Neurosciences, Volume 12, Issue 2 (April), 2005].

There remains a need for novel effective drugs in the treatment of Parkinson's disease.

### Aims of the Invention

The invention aims to solve the technical problem of providing an active ingredient for the treatment of Parkinson's disease.

The invention also aims to solve the technical problem of providing an active ingredient for an efficient treatment of Parkinson's disease, especially in human patients.

The invention also aims to solve the technical problem of providing an active ingredient that improves prior art methods for the treatment of Parkinson's disease.

The invention aims to provide an efficient treatment for Parkinson's disease at an appropriate dose, route of administration, and daily intake.

### SUMMARY OF THE INVENTION

Mast cells, which are found on both sides of the blood-brain barrier (BBB), play an important role in sustaining the inflammatory network [Theoharides TC. Mast cells and stress—a psychoneuroimmunological perspective. J Clin Psychopharmacol. 2002 Apr;22(2):103-8] [Stassen M, et al. Mast cells and inflammation. Arch Immunol Ther Exp (Warsz). 2002; 50(3):179-85] [Kinet JP. The essential role of mast cells in orchestrating inflammation. Immunol Rev. 2007 Jun;217:5-7]. Moreover, it has been shown that mast cells are able to cross the BBB and their numbers may rapidly increase in response to physiological manipulations [Nautiyal K, et al. Brain mast cells link the immune system to anxiety-like behavior. Proc Natl Acad Sci USA. 2008 November; 18; 105(46): 18053-18057] [Theoharides TC, et al. Critical role of mast cells in inflammatory diseases and the effect of acute stress. J Neuroimmunol. 2004 Jan; 146 (1-2):1-12] [Silverman AJ, et al. Mast cells migrate from blood to brain. J Neurosci 2000, 20:401-408]. Hence, mast cells may actively participate in the pathogenesis of Parkinson's disease, in part because they release large amounts of proinflammatory mediators that sustain the inflammatory network of the central nervous system.

Perivascular localized mast cells secrete numerous vasoactive molecules that regulate BBB permeability [Secor VH, et al. Mast cells are essential for early onset and severe disease in a murine model of multiple sclerosis. J Exp Med. 2000 Mar 6;191(5):813-22] [Esposito P, et al. Corticotropin-releasing hormone and brain mast cells regulate blood-brain-barrier permeability induced by acute stress. J Pharmacol Exp Ther. 2002;303:1061-1066] [Esposito P, et al. Acute stress increases permeability of the blood-brain-barrier through activation of brain mast cells. Brain Res. 2001 Jan 5;888(1):117-127] [Zhuang X, et al. Brain mast cell degranulation regulates blood-brain barrier. J Neurobiol. 1996 Dec;31(4):393-403]. Inhibition of mast cell mediators and apoptosis of mast cells localized at the BBB would effectively reduce BBB permeability, thereby reinforcing its integrity and stemming the accumulation of exogenous damaging factors in the brain.

Increased permeability of the BBB (Brain-Blood-Barrier) is an established event associated with clinical and pathological signs of neurodegenerative diseases. Indeed, BBB dysfunction is also identified as being a factor in Parkinson's disease [Kortekaas R, Leenders KL, van Oostrom JC, Vaalburg W, Bart J, Willemsen AT, Hendrikse NH. Blood-brain barrier dysfunction in parkinsonian midbrain in vivo. Ann Neurol. 2005 Feb; 57(2):176-9].

It has been found according to the present invention that a tyrosine kinase inhibitor or mast cell inhibitor, in particular masitinib or a pharmaceutically acceptable salt or solvate thereof, is useful in the treatment of Parkinson's disease.

In one embodiment, said tyrosine kinase inhibitor or mast cell inhibitor is administered in combination with at least one pharmaceutically active ingredient. Said pharmaceutically active ingredient is preferably active in the treatment of Parkinson's disease. Such pharmaceutically active ingredient is preferably chosen from the group consisting of: levodopa, carbidopa-levodopa, dopamine agonists, monoamine oxidase B (MAO-B) inhibitors, catechol-O-methyl transferase (COMT) inhibitors, NMDA receptor antagonists, acetylcholinesterase inhibitors, and mixture thereof.

The present invention thus relates to a method for the treatment of Parkinson's disease in a mammal, and especially a human patient, wherein said method comprises administering to a human patient in need thereof, a tyrosine kinase inhibitor or mast cell inhibitor, especially masitinib or a pharmaceutically acceptable salt or solvate thereof, optionally combined with at least one pharmaceutically active ingredient.

The invention also relates to a pharmaceutical composition or kit comprising a tyrosine kinase inhibitor or mast cell inhibitor, in particular masitinib or a pharmaceutically acceptable salt or solvate thereof, and at least one other pharmaceutically active ingredient, for use in a method for the treatment of Parkinson's disease as defined according to the present invention.

The invention also relates to the use a tyrosine kinase inhibitor or mast cell inhibitor, in particular masitinib or a pharmaceutically acceptable salt or solvate thereof, for the preparation of a medicament, or a pharmaceutical composition, for the treatment of Parkinson's disease, optionally in combination with at least one other pharmaceutically active ingredient.

### DESCRIPTION OF THE INVENTION

Tyrosine kinases are receptor type or non-receptor type proteins, which transfer the terminal phosphate of ATP to tyrosine residues of proteins thereby activating or inactivating signal transduction pathways. These proteins are known to be involved in many cellular mechanisms, which in case of disruption, lead to disorders such as abnormal cell proliferation and migration as well as inflammation. A tyrosine kinase inhibitor is a drug that inhibits tyrosine kinases, thereby interfering with signaling processes within cells. Blocking such processes can stop the cell growing and dividing or inhibit cell activity.

In one embodiment, the tyrosine kinase inhibitor of the invention has the following formula [A]:

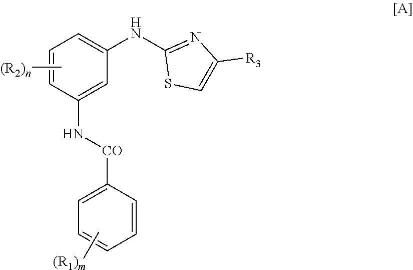

wherein R₁ and R₂, are selected independently from hydrogen, halogen, a linear or branched alkyl, cycloalkyl group containing from 1 to 10 carbon atoms, trifluoromethyl, alkoxy, cyano, dialkylamino, and a solubilizing group,
m is 0-5 and n is 0-4;
the group R3 is one of the following:
(i) an aryl group such as phenyl or a substituted variant thereof bearing any combination, at any one ring position, of one or more substituents such as halogen, alkyl groups containing from 1 to 10 carbon atoms, trifluoromethyl, cyano and alkoxy;
(ii) a heteroaryl group such as 2, 3, or 4-pyridyl group, which may additionally bear any combination of one or more substituents such as halogen, alkyl groups containing from 1 to 10 carbon atoms, trifluoromethyl and alkoxy;
(iii) a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy; or a pharmaceutically acceptable salt or solvate thereof.

Tyrosine kinase inhibitors of formula [A] can preferably be used as c-Kit inhibitors.

Unless otherwise specified, the below terms used herein are defined as follows:

As used herein, the term an “aryl group” means a monocyclic or polycyclic-aromatic radical comprising carbon and hydrogen atoms. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents. In one embodiment, the aryl group is a monocyclic ring, wherein the ring comprises 6 carbon atoms, referred to herein as “(C₆)aryl”.

As used herein, the term “alkyl group” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3 ,3-dimtheylpentyl, 3 ,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more substituents.

As used herein, the term “alkoxy” refers to an alkyl group which is attached to another moiety by an oxygen atom. Examples of alkoxy groups include methoxy, isopropoxy, ethoxy, tert-butoxy, and the like. Alkoxy groups may be optionally substituted with one or more substituents.

As used herein, the term “heteroaryl” or like terms means a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

The term “heterocycle” as used herein, refers collectively to heterocycloalkyl groups and heteroaryl groups.

As used herein, the term “heterocycloalkyl” means a monocyclic or polycyclic group having at least one heteroatom selected from O, N or S, and which has 2-11 carbon atoms, which may be saturated or unsaturated, but is not aromatic. Examples of heterocycloalkyl groups include (but are not limited to): piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein the term “substituent” or “substituted” means that a hydrogen radical on a compound or group is replaced with any desired group that is substantially stable to reaction conditions in an unprotected form or when protected using a protecting group. Examples of preferred substituents are those found in the exemplary compounds and embodiments disclosed herein, as well as halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH₂; OCH₂CONH₂; NH₂; SO₂NH2; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl; an alkenyl; an alkynyl; an cycloalkyl; an cycloalkenyl; a heterocycloalkyl; an aryl; a heteroaryl; an aralkyl; a heteraralkyl; a haloalkyl; —C(O)NR₁₁R₁₂, —NR₁₃C(O)R₁₄, a halo; —OR₁₃; cyano, nitro; a haloalkoxy; —C(O)R₁₃; —NR₁₁R₁₂; —SR₁₃; —C(O)OR₁₃; —OC(O)R₁₃; —NR₁₃C(O)NR₁₁R₁₂; —OC(O)NR₁₁R₁₂; —NR₁₃C(O)OR₁₄; —S(O)rR₁₃; —NR₁₃S(O)rR₁₄; —OS(O)rR₁₄; S(O)rNR₁₁R₁₂; —O; —S; and —N—R₃; wherein r is 1 or 2; R₁₁ and R₁₂, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₁₁ and R₁₂ taken together with the nitrogen to which they are attached is optionally substituted heterocycloalkyl or optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl.

In certain embodiments, the term “substituent” or the adjective “substituted” refers to a solubilizing group.

The term “solubilizing group” means any group which can be substantially ionized and that enables the compound to be soluble in a desired solvent, such as, for example, water or water-containing solvent. Furthermore, the solubilizing group can be one that increases the compound or complex's lipophilicity. Typically, the solubilizing group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid. For example, by “solubilizing group” it is referred herein to one of the following:
an alkyl, cycloalkyl, aryl, heretoaryl group comprising either at least one nitrogen or oxygen heteroatom or which group is substituted by at least one amino group or oxo group;
an amino group which may be a saturated cyclic amino group which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl;
one of the structures a) to i) shown below, wherein the wavy line and the arrow line correspond to the point of attachment to core structure of Formula [A]:

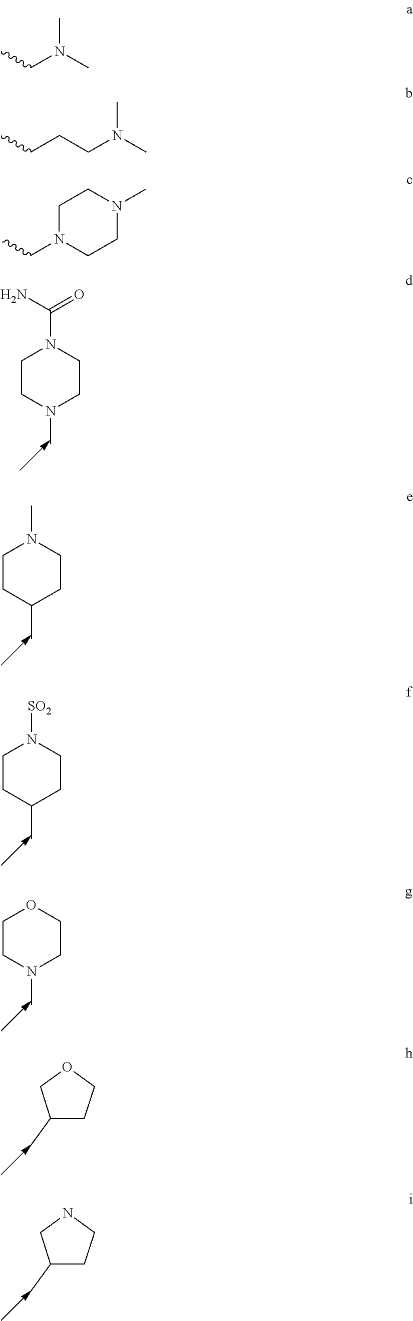

The term “cycloalkyl” means a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

The term “halogen” means —F, —Cl, —Br or —I.

In a particular embodiment, the tyrosine kinase inhibitor of the invention has general formula [B],

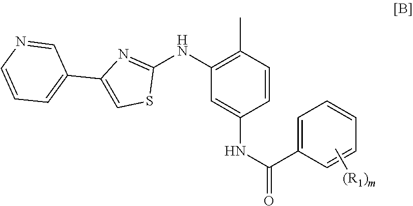

wherein:

R₁is selected independently from hydrogen, halogen, a linear or branched alkyl, cycloalkyl group containing from 1 to 10 carbon atoms, trifluoromethyl, alkoxy, amino, alkylamino, dialkylamino, solubilizing group, and m is 0-5.

In one embodiment, the tyrosine kinase inhibitor or mast cell inhibitor is masitinib or a pharmaceutically acceptable salt thereof, more preferably masitinib mesilate.

Masitinib is a c-Kit/PDGFR/Lyn inhibitor with a potent anti mast cell action. Masitinib is therefore a mast cell inhibitor.

New potent and selective tyrosine kinase inhibitors are 2-(3-aminoaryl)amino-4-aryl-thiazoles described in AB Science's PCT application WO 2004/014903.

Masitinib (AB1010) is a small molecule drug, selectively inhibiting specific tyrosine kinases such as c-Kit, PDGFR, Lyn, and Fyn without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) [Dubreuil et al., 2009, PLoS ONE 2009.4(9):e7258] [Davis et al., Nat Biotechnol 2011, 29(11): 1046-51]. The chemical name for masitinib is 4-(4-methylpiperazin-l-ylmethyl)-N- [4-methyl-3 -(4-pyridin-3ylthiazol-2-ylamino) phenyl]benzamide—CAS number 790299-79-5, and the structure is shown below. Masitinib was first described in U.S. Pat. No. 7,423,055 and EP1525200B1. A detailed procedure for the synthesis of masitinib mesilate is given in WO 2008/098949.

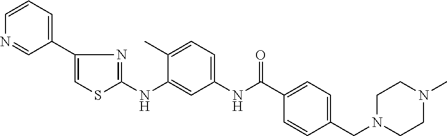 [SMILES: Cc1ccc(NC(=O)c2ccc(CN3CCN(C)CC3)cc2)cc1Nc1nc(-c2cccnc2)cs1]

Masitinib' s main kinase target is c-Kit, for which it has been shown to exert a strong inhibitory effect on wild-type and juxtamembrane-mutated c-Kit receptors, resulting in cell cycle arrest and apoptosis of cell lines dependent on c-Kit signaling [Dubreuil et al., 2009, PLoS ONE, 4(9):e7258]. In vitro, masitinib demonstrated high activity and selectivity against c-Kit, inhibiting recombinant human wild-type c-Kit with a half inhibitory concentration (IC50) of 200±40 nM and blocking stem cell factor-induced proliferation and c-Kit tyrosine phosphorylation with an IC50 of 150±80 nM in Ba/F3 cells expressing human or mouse wild-type c-Kit. In addition to its anti-proliferative properties, masitinib can also regulate the activation of mast cells through its targeting of Lyn and Fyn, key components of the transduction pathway leading to IgE induced degranulation [Gilfillan et al., 2006, Nat Rev Immunol, 6:218-230] [Gilfillan et al., 2009, Immunological Reviews, 228:149-169]. This can be observed in the inhibition of FcϵRI-mediated degranulation of human cord blood mast cells [Dubreuil et al., 2009, PLoS ONE;4(9):e7258]. Masitinib is also an inhibitor of PDGFR α and β receptors. Recombinant assays show that masitinib inhibits the in vitro protein kinase activity of PDGFR-α and β with IC50 values of 540±60 nM and 800±120 nM. In Ba/F3 cells expressing PDGFR-α, masitinib inhibited PDGF-BB-stimulated proliferation and PDGFR-α tyrosine phosphorylation with an IC50 of 300±5 nM.

The present invention relates to a method for the treatment of Parkinson's disease in a mammal, and especially a human patient, wherein said method comprises administering to a human patient in need thereof, a tyrosine kinase inhibitor or mast cell inhibitor, especially masitinib or a pharmaceutically acceptable salt or solvate thereof, optionally combined with at least one pharmaceutically active ingredient.

The present invention relates to a method for the treatment of Parkinson's disease wherein said method comprises administering to a mammal in need thereof, at least one tyrosine kinase inhibitor or mast cell inhibitor.

In one embodiment, said tyrosine kinase inhibitor or mast cell inhibitor is administered to a human patient.

In one embodiment, said tyrosine kinase inhibitor is an inhibitor of kinase activity selected from the tyrosine kinase activity of: c-Kit, Lyn, Syk, Btk and Fyn.

In one embodiment, said tyrosine kinase inhibitor is an inhibitor of kinase activity selected from the tyrosine kinase activity of: c-Kit and Lyn.

In one embodiment, said tyrosine kinase inhibitor is a selective inhibitor of mast cell function.

In one embodiment, said mast cell inhibitor is masitinib or a pharmaceutically acceptable salt or solvate thereof, more preferably masitinib mesilate.

In another embodiment, said mast cell inhibitor is imatinib (STI571, Novartis), more preferably imatinib mesilate. Therefore, in a particular embodiment, the invention relates to a method for the treatment of Parkinson's disease in a mammal, and especially a human patient, comprising the administration of an effective amount of the compound known in the art as imatinib (STI571, CGP57148B): 4-[(4-Methyl-1-piperazinyl) methyl]-N-(4-methyl-3-{[4-(3-pyridinyl)-2-pyrimidinyl]amino}phenyl)benzamide. The preparation of this compound is described in example 21 of EP 564 409 and the form, which is particularly useful is described in WO 99/03854.

In another embodiment, the mast cell inhibitor can be selected from: midostaurin (PKC412; Novartis), dasatinib (BMS354825; Bristol-Myers Squibb), sunitinib (SU11248; Pfizer), axitinib (AG013736; Pfizer), pazopanib (GlaxoSmithKline), toceranib (SU11654; Pfizer), BLU-285 (Blueprint Medicines), bosutinib (SKI-606; Pfizer), ibrutinib (PCI-32765; Pharmacyclics), LAS189386 (Almirall R&D Center), DP-2618 (Deciphera Pharmaceuticals), fostamatinib (R788; Rigel), and cromolyn sodium.

In another embodiment, the mast cell inhibitor is chosen from the group consisting of: masitinib, imatinib, cromolyn sodium, midostaurin, BLU-285, bosutinib, ibrutinib, LAS189386, DP-2618, fostamatinib, dasatinib, sunitinib, axitinib, pazopanib, and toceranib, or pharmaceutically acceptable salts or solvates thereof.

In one embodiment, said tyrosine kinase inhibitor or mast cell inhibitor is administered in combination with at least one pharmaceutically active ingredient. Said pharmaceutically active ingredient is preferably active in the treatment of Parkinson's disease. Such pharmaceutically active ingredient is preferably chosen from the group consisting of: levodopa, carbidopa-levodopa, dopamine agonists, monoamine oxidase B (MAO-B) inhibitors, catechol-O-methyl transferase (COMT) inhibitors, NMDA receptor antagonists, acetylcholinesterase inhibitors, and mixture thereof. The dopamine agonist is preferably chosen from: bromocriptine, pergolide, pramipexole, ropinirole, piribedil, cabergoline, apomorphine and lisuride. The MAO-B inhibitor is preferably chosen from: safinamide, selegiline and rasagiline. The COMT inhibitor is preferably chosen from: entacapone and tolcapone. The NMDA receptor agonist is preferably chosen from: amantadine and memantine. The acetylcholinesterase inhibitor is preferably chosen from: rivastigmine, donepezil, and galantamine. Therefore, in embodiment, said pharmaceutically active ingredient is chosen from the group consisting of: levodopa, carbidopa-levodopa, bromocriptine, pergolide, pramipexole, ropinirole, piribedil, cabergoline, apomorphine lisuride, safinamide, selegiline, rasagiline, entacapone, tolcapone, amantadine, memantine, rivastigmine, donepezil, galantamine, and mixture thereof.

In one embodiment, said tyrosine kinase inhibitor or mast cell inhibitor, in particular masitinib or a pharmaceutically acceptable salt or solvate thereof, is administered at a daily dose of between 1.5 to 9.0 mg/kg/day; for example, 1.5, 3.0, 4.5, 6.0, 7.5, or 9.0 mg/kg, more preferably 3.0, 4.5 or 6 mg/kg/day (mg per kg bodyweight per day).

In one embodiment said tyrosine kinase inhibitor or mast cell inhibitor, in particular masitinib or a pharmaceutically acceptable salt or solvate thereof, is dose escalated by increments of 1.5 mg/kg/day to reach a maximum of 9.0 mg/kg/day, more preferably 6 mg/kg/day. Each dose escalation is subjected to toxicity controls with an absence of any toxicity events permitting dose escalation to occur.

In one embodiment dose escalation of said tyrosine kinase inhibitor or mast cell inhibitor, in particular masitinib or a pharmaceutically acceptable salt or solvate thereof, occurs at any time-point after at least 4 weeks after the initial dose has been administered and prior to 26 weeks after the initial dose has been administered; for example, at week-4, week-8, week-12, week-16, week-20, or week-24. As an example, said tyrosine kinase inhibitor or mast cell inhibitor, in particular masitinib or a pharmaceutically acceptable salt or solvate thereof, is initially administered per os, preferably in two daily intakes, at a dose of 3 mg/kg/day during 4 weeks, then 4.5 mg/kg/day during 4 weeks, and then 6 mg/kg/day thereafter. In another example, masitinib or a pharmaceutically acceptable salt or solvate thereof is initially administered per os, preferably in two daily intakes, at a dose of 4.5 mg/kg/day during 12 weeks, and then 6 mg/kg/day thereafter.

Any dose indicated herein refers to the amount of active ingredient as such, not to its salt form.

Given that the masitinib dose in mg/kg/day used in the described dose regimens refers to the amount of active ingredient masitinib, compositional variations of a pharmaceutically acceptable salt of masitinib mesilate will not change the said dose regimens.

In one embodiment, said tyrosine kinase inhibitor or mast cell inhibitor, in particular masitinib or a pharmaceutically acceptable salt or solvate thereof is administered orally.

In one embodiment, said tyrosine kinase inhibitor or mast cell inhibitor, in particular masitinib or a pharmaceutically acceptable salt or solvate thereof is administered once or twice a day.

In one embodiment, said tyrosine kinase inhibitor or mast cell inhibitor, in particular masitinib or a pharmaceutically acceptable salt or solvate thereof, is administered in combination with said at least one pharmaceutically active ingredient in a combined preparation for simultaneous, separate, or sequential use.

The invention also relates to a tyrosine kinase inhibitor or mast cell inhibitor, in particular masitinib or a pharmaceutically acceptable salt or solvate thereof, as defined according to the present invention, for use in a treatment of Parkinson's disease.

The invention also relates to a tyrosine kinase inhibitor or mast cell inhibitor, in particular masitinib or a pharmaceutically acceptable salt or solvate thereof, as defined according to the present invention, for use in a treatment of Parkinson's disease, in combination with at least pharmaceutically active ingredient, preferably chosen from the group consisting of: levodopa, carbidopa-levodopa, bromocriptine, pergolide, pramipexole, ropinirole, piribedil, cabergoline, apomorphine, lisuride, safinamide, selegiline, rasagiline, entacapone, tolcapone, amantadine, memantine, rivastigmine, donepezil, galantamine, and mixture thereof.

The invention also relates to a pharmaceutical composition or kit comprising a tyrosine kinase inhibitor or mast cell inhibitor, in particular masitinib or a pharmaceutically acceptable salt or solvate thereof, for use in a method for the treatment of Parkinson's disease as defined according to the present invention.

In one embodiment, the pharmaceutical composition for use in a method for the treatment of Parkinson's disease according to the present invention comprises a mast cell inhibitor, preferably masitinib or a pharmaceutically acceptable salt or solvate thereof, in combination with one or more pharmaceutically acceptable excipients.

The invention also relates to a pharmaceutical composition or kit comprising a tyrosine kinase inhibitor or mast cell inhibitor, in particular masitinib or a pharmaceutically acceptable salt or solvate thereof, and at least one other pharmaceutically active ingredient, preferably chosen from the group consisting of: levodopa, carbidopa-levodopa, bromocriptine, pergolide, pramipexole, ropinirole, piribedil, cabergoline, apomorphine, lisuride, safinamide, selegiline, rasagiline, entacapone, tolcapone, amantadine, memantine, rivastigmine, donepezil, galantamine, and mixture thereof.

The invention also relates to the use a tyrosine kinase inhibitor or mast cell inhibitor, in particular masitinib or a pharmaceutically acceptable salt or solvate thereof, for the preparation of a medicament, or a pharmaceutical composition, for the treatment of Parkinson's disease, optionally in combination with at least one other pharmaceutically active ingredient, preferably chosen from the group consisting of: levodopa, carbidopa-levodopa, bromocriptine, pergolide, pramipexole, ropinirole, piribedil, cabergoline, apomorphine, lisuride, safinamide, selegiline, rasagiline, entacapone, tolcapone, amantadine, memantine, rivastigmine, donepezil, galantamine, and mixture thereof.

The terms “as defined according to the invention” refer to any embodiments or aspects of the invention alone or in combination without limitation, including any preferred embodiments and variants, including any embodiments and features relating to said tyrosine kinase inhibitor or mast cell inhibitor, in particular masitinib or a pharmaceutically acceptable salt or solvate thereof, the method of treatment of Parkinson's disease, pharmaceutical compositions and any combination with other pharmaceutically active ingredient(s), preferably chosen from the group consisting of: levodopa, carbidopa-levodopa, bromocriptine, pergolide, pramipexole, ropinirole, piribedil, cabergoline, apomorphine, lisuride, safinamide, selegiline, rasagiline, entacapone, tolcapone, amantadine, memantine, rivastigmine, donepezil, galantamine, and mixture thereof. “Masitinib ” designates also a pharmaceutically acceptable salt or solvate thereof, especially masitinib mesilate, even when not explicitly stated.

The tyrosine kinase inhibitor or mast cell inhibitor and the optional at least one pharmaceutically active ingredient, are administered in a dosage regimen that comprises a therapeutically effective amount.

In relation to the present invention, the term “treatment” (and its various grammatical forms) refers to preventing, curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease.

Advantageously, the use or method comprises a long term administration of an effective amount of said tyrosine kinase inhibitor or mast cell inhibitor, especially masitinib or a pharmaceutically acceptable salt or solvate thereof, over more than 3 months, preferably more than 6 months.

In one embodiment, the use or method comprises administering said tyrosine kinase inhibitor or mast cell inhibitor, especially masitinib or a pharmaceutically acceptable salt or solvate thereof, as first, second or third-line treatment of Parkinson's disease in a mammal, and especially a human patient.

As is known to the person skilled in the art, various forms of excipients can be used adapted to the mode of administration and some of them can promote the effectiveness of the active molecule, e.g. by promoting a release profile rendering this active molecule overall more effective for the treatment desired.

The pharmaceutical compositions of the invention are thus able to be administered in various forms, more specially for example in an injectable, pulverizable or ingestible form, for example via the intramuscular, intravenous, subcutaneous, intradermal, oral, topical, rectal, vaginal, ophthalmic, nasal, transdermal or parenteral route. A preferred route is oral administration. The present invention notably covers the use of a compound according to the present invention for the manufacture of pharmaceutical composition.

Such medicament can take the form of a pharmaceutical composition adapted for oral administration, which can be formulated using pharmaceutically acceptable carriers well known in the art in suitable dosages. Such carriers enable the pharmaceutical compositions to be formulated as tablets, pills, dragees, capsules, liquids, gels, syrups, slurries, suspensions, and the like, for ingestion by the patient. In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

According to a particular embodiment, the composition of the invention is an oral composition.

In one embodiment, compositions according to the invention may be in the form of tablets.

In one embodiment, composition according to the invention may comprise from 50 to 500 mg of said tyrosine kinase inhibitor or mast cell inhibitor, especially masitinib or a pharmaceutically acceptable salt or solvate thereof. More particularly, the composition may comprise from 100 to 500 mg of said tyrosine kinase inhibitor or mast cell inhibitor, especially masitinib or a pharmaceutically acceptable salt or solvate thereof, for example, 100, 200, 300, 400, or 500 mg.

The present invention is further illustrated by means of the following examples.

The data presented in these examples, and also in parts of the patent Description, are in part taken from preliminary analysis and as such represent a close approximation to the final, validated dataset.

### EXAMPLES

### EXAMPLE 1: EFFECT OF MASITINIB ON PARKINSON'S DISEASE IN MICE MODEL

One of the established experimental mouse model of idiopathic Parkinson's disease is a systemic administration of 1-methyl-4-phenyl-1,2,3,6-tetrahydropyridine (MPTP). This toxin produces the same marked depletion of striatal dopamine, its metabolites and terminals, and destruction of dopaminergic neurons in the substantia nigra.

The implication of mast cell in the development of Parkinson's disease is not well characterized. Nevertheless, some studies have shown that the amount of histamine (a molecule released by mast cells) was increased in the brain of a patient suffering from Parkinson's disease. To test the role of these inflammatory cells in this pathology, masitinib, a tyrosine kinase inhibitor, which specifically targets mast cells survival, proliferation and activation, was used. To analyze the putative neuroprotective effect of a drug acting on mast cells in an animal model of Parkinson's disease (PD), the MPTP mouse model of PD was used. The survival of dopaminergic neurons was studied by the characterization of the tyrosine hydroxylase expression.

The present work was undertaken to evaluate the effect of masitinib in a model of Parkinson's disease induced by MPTP in the mouse.

Mice were treated with masitinib or solvant alone starting day 7 before intoxication. Each treated group included 10 animals for the MPTP intoxicated groups and 5 animals for the controls. The loss of tyrosine hydroxylase expression, indicative of dopaminergic neuron destruction, was analyzed by western blotting, immunohistochemistry and ELISA assay.

### 1.1. Materials and Methods

### 1.1.1. Experimental Items

40 outbred, C57BL/6 male mice, 23-26 g (January; France);
1-Methyl-4-Phenyl-1,2,3,6-TetrahydroPyridine (MPTP) (Cat #: M0896; Sigma Aldrich; France);
Masitinib (AB Science);
Tween 80 (Cat #: P8074; Sigma Aldrich; France);
1,2 propanediol (Cat #: 398039; Sigma Aldrich; France);
0.9% saline, sterile (B. Braun);
Polyclonal Antibody Anti-Tyrosine Hydroxylase (Cat #AB152; Millipore; France);
Antibody biotinylated goat anti rabbit IgG (Cat #111-65-003, Jakson immunoresearch);
Streptavidin peroxidase complex (Cat #E2886, Sigma Aldrich, France);
Z-Lys-SBZL substrate solution (Cat #C3641, Sigma Aldrich, France);
DTNB (Cat #D8130, Sigma Aldrich, France);
Abcys Histogreen substrate kit (Cat #E109, AbCys);
Amplex Red Monoamine Oxidase Assay kit (Cat #A12214, Invitrogen).

### 1.1.2. Preparation of Masitinib

400 mg of masitinib was stored at room temperature. The day before treatment starts, masitinib was dissolved to 6.25 mg/ml in 10% Tween 80; 10% Isopropanediol; 80% water solution, and thereafter aliquoted and stored at −20′C. This stock solution was defrosted before treatment of mice. The mice were treated orally by gavage with 0.1 ml corresponding to 25 mg/kg in a 24 g mouse.

### 1.1.3. Preparation of MPTP Solution

One ampoule of 100 mg MPTP was dissolved in 20 ml NaCl 0.9% to obtain 20 mg/kg of MPTP in 100 μl injected by mouse.

### 1.1.4. Inoculation of Mice

C57BL/6J mice are intoxicated with MPTP using an acute protocol, as described hereafter. On the day of MPTP exposure each mouse received 4 injections of MPTP solution (20 mg/kg) at 2-hour intervals for 1 day.

### 1.1.5. Treatment of Mice

Masitinib was administered by oral route in a volume of 0.1 mL in the morning and in the afternoon. Dosing was done 2 times per day at 6-hour interval starting day 7 before intoxication and stopping 7 days after MPTP administration. On day 14, mice were deeply anesthetized with 12.5 mg/kg Xylasine (Bayer, France), 192 mg/kg Ketamine (Mérial, France) diluted in physiological saline solution. Before brain extraction mice were perfused with 0.9% NaCl 2.6 mM EDTA solution.

During the period of experiment, the animals were treated either with masitinib twice a day at 30 mg/kg (single-agent, masitinib control group) or at 5 mg/kg (test group) or with solvent (control group) using per os administration. The treatment started 7 days before MPTP administration and continued 7 days after.

### 1.1.6. Tissue and protein samples

After mouse sacrifice and dissection, the left brain was submerged in 4% paraformaldehyde in PBS (phosphate buffered saline) solution for paraffin embedding and then cut into 5 μM-thick coronal section. And the right brain was immediately frozen in nitrogen liquid for protein analysis. The proteins were extracted with the homogenizer ultra turrax IKA-T10 basic system and quantified using the BCA test (bicinchoninic acid assay).

### 1.1.7. Tyrosine hydroxylase western blotting

30 μg of total brain protein were used for western blot analysis using 1:1000 dilution anti-tyrosine hydroxylase rabbit polyclonal antibody (Cat #AB152, Millipore) followed by 1:10000 horseradish peroxidase-conjugated anti-rabbit antibody (Jakson ImmunoResearch). Antibody to actin (Cat #A5316, Sigma Aldrich) were used as loading control followed by 1:10000 horseradish peroxidase-conjugated anti-mouse antibody (Jakson ImmunoResearch). Immunoreactive bands were detected using enhanced chemiluminescent reagents (GE Healthcare, Amersham, UK). Signal intensity was calculated using the Image J software.

### 1.1.8. Tyrosine Hydroxylase ELISA Assay

The microliter plate wells were coated overnight at 4° C. with 5μg of total brain protein extract. All unbound sites were blocked with a blocking buffer 10% FCS in PBS 1 hour at room temperature. Then, the anti-tyrosine hydroxylase rabbit polyclonal antibody, diluted 1:1000 was added for 2 hours at room temperature. After rinsing with PBS, the wells were incubated with biotinylated goat anti-rabbit IgG (1:250) for 1 hour at room temperature (Cat #111-65-003, Jakson immunoresearch) and followed by incubation with a streptavidin peroxidase complex for 30 minutes (1:250) at room temperature (Cat #E2886, Sigma Aldrich). Peroxidase staining was revealed using the manufacturer's instructions of the TMB ELISA kit detection.

### 1.1.9. Tyrosine Hydroxylase Immunohistochemistry

The 5-μM-thick coronal sections were deparaffinized and hydrated, Endogenous peroxydase were inhibited with a dual endogenous enzyme block solution (Cat #S2003, DakoCytonation). Non specific protein binding was blocked with 1% BSA in PBS, pH=7,4. Sections were incubated overnight at 4° C. with anti-tyrosine hydroxylase rabbit polyclonal antibody, diluted 1:1000 (Cat #AB152, Millipore). After rinsing with PBS, the sections were incubated with biotinylated goat anti-rabbit IgG (1:500) for 30 minutes at room temperature (Cat #111-65-003, Jakson immunoresearch) followed by incubation with a streptavidin peroxidase complex for 30 minutes (1:500) at room temperature (Cat #E2886, Sigma Aldrich). Peroxidase staining was obtained using the AbCys Histogreen substrate kit (Cat #E109, AbCys) and counterstained with a Neutral Red solution. After dehydration the sections were cover-slipped with Eukitt®.

### 1.1.10. Tryptase Enzymatic Colorimetric Assay

5 μg of total brain protein extract were added in microliter plate wells and the quantity of tryptase enzyme measured by densitometry at 410 nm after addition of Z-Lys-SBZL substrate solution (Cat #C3641, Sigma Aldrich) and DTNB (Cat #D8130, Sigma Aldrich).

### 1.1.11. Monoamine Oxidase Fluorometric Assay

5 μg of total brain protein extract were added in microliter plate wells and the monoamine oxidase activity measured using manufacturer's instructions (Cat #A12214, Invitrogen) by fluorometric method using excitation at 545 nm and using emission detection at 590 nm.

### 1.1.12. Statistical Comparison

Statistical comparison of two selected groups was done with Mann Whitney test and comparison of multiple groups was done with ANOVA and Turkey's multiple comparison tests.

### 1.2. Results

No mortality was observed 24 hours after treatment by the MPTP. Brain samples were collected after mice anesthesia and sacrifice.

The tyrosine hydroxylase expression was detected from total brain protein extract both by western blotting and by ELISA assays (Table 1 and Table 2, respectively).

A decrease of the tyrosine hydroxylase expression, indicating dopaminergic neuron destruction, was detected after MPTP treatment relative to the control group. This result validates the protocol.

TABLE 1
Relative expression of Tyrosine Hydroxylase
in total brain protein extract using Western Blotting assay
after treatment by MPTP and masitinib.

| | Relative average expression |
|---|---|
| Treatment | of tyrosine hydroxylase |
| Control | 100 |
| Masitinib 2 × 30 mg/kg | 90 |
| MPTP | 77 |
| MPTP + Masitinib 2 × 5 mg/kg | 91 |

TABLE 2
Tyrosine Hydroxylase in total brain protein extract
using ELISA assay after treatment
by MPTP and masitinib.

| | Mean expression of |
|---|---|
| Treatment | tyrosine hydroxylase |
| Control | 0.275 |
| Masitinib 2 × 30 mg/kg | 0.225 |
| MPTP | 0.175 |
| MPTP + Masitinib 2 × 5 mg/kg | 0.215 |

A significant decrease of tyrosine hydroxylase expression was detected after MPTP treatment with respect to the control group. The treatment with masitinib alone did not modify significantly the basal expression of tyrosine hydroxylase compared to the control. The treatment with 2 x 5 mg/kg of masitinib compound in the MPTP group showed a significant attenuation of tyrosine hydroxylase destruction. This positive effect of masitinib administrated at this concentration was observed using the in situ staining of tyrosine hydroxylase sustaining the tyrosine hydroxylase quantification assay. This result was reproducible with western blotting, ELISA, and immunohistochemistry assays (performed on 5 μM-thick coronal sections). These results therefore demonstrate a neuroprotective effect of masitinib in mice receiving masitinib treatment at 5 mg/kg twice per day and more generally proof-of-concept of a neuroprotective effect for inhibitors of mast cell function.

Several drugs are effective in the MPTP mouse model by virtue of their inhibitory activity on the enzyme Monoamine oxidase B (MAO-B); for example, peroxisome agonist of proliferator-activated receptor or PPAR agonist. Indeed, the MAO-B enzyme transforms the MPTP into an active neurotoxic metabolite 1-methyl-4-phenylpyridinium (MPP+) which induces dopaminergic neuronal destruction. In order to evaluate if the mechanism of protection of masitinib observed against MPTP induced toxicity was due to the MAO-B inhibition, the MAO enzymatic activity from total brain protein extract was quantified (Table 3).

TABLE 3
Monoamine oxidase activity detected by fluorometric
method from total brain protein extract after
treatment by MPTP and masitinib.

| | Monoamine oxidase |
|---|---|
| Treatment | fluorometric assay |
| Control | 100 |
| MPTP | 108 |
| MPTP + Masitinib 2 × 5 mg/kg | 115 |

In all mice groups treated with MPTP alone or in combination with masitinib, the activity of monoamine oxidase was maintained with respect to the control group. Hence, one may conclude that the observed treatment-effect with 2×5 mg/kg masitinib is likely due to an anti-inflammatory action rather than MAO-B inhibition.

### 1.3. Conclusions

The results showed masitinib protection against the decrease expression of tyrosine hydroxylase. This was confirmed by using different methods of analysis such as Western blot and ELISA test. These results indicate that masitinib might offer a protective role in the development of idiopathic Parkinson's disease. Moreover, no modification of monoamine oxidase enzymatic activity was observed, which appeared to show that the mechanism of protection of masitinib observed against MPTP induced toxicity was due to an anti-inflammatory action.

These preclinical findings provide proof-of-concept of masitinib' s neuroprotective potential. Masitinib is an effective targeted therapy against mast cells, exerting a direct proapoptotic, anti-migratory, and anti-activation action [Dubreuil et al., 2009, PLoS ONE;4(9):e7258], thus, indirectly controlling the array of proinflammatory and vasoactive mediators these cells can release. Given that the neural pool of mast cells is influenced by their ability to rapidly cross the BBB, inhibition of mast cells peripheral to the BBB could therefore impact on the main pathological features of Parkinson's disease. In conclusion, this study showed that masitinib, a potent and selective inhibitor mast cell activity, may be used as a potential treatment of Parkinson's disease.

### EXAMPLE 2: CLINICAL STUDY PROTOCOL

Study design: Multicenter, randomized, double-blind, placebo-controlled, parallel group, phase 2 study to compare the efficacy and safety of masitinib in the treatment of patients suffering from Parkinson's disease.

Diagnosis: Non-demented patients with idiopathic Parkinson's disease (PD) and cognitive impairment.

Study treatment: masitinib 100 and 200 mg tablets.

Associated product: Placebo, matching 100 mg and 200 mg tablets.

Duration of treatment: 48 weeks of study treatment with possible extension.

The objective is to compare the efficacy and safety of masitinib in cognitively impaired but non-demented Parkinson's disease patients. Eligible patients will be treated during 48 weeks and patients will be proposed to enter a double-blind extension phase.

At week 48, patients will be allowed to continue their treatment at the same dose level providing that the benefit/risk balance is still in favor of treatment continuation according to the investigator resulting in an absence of progression and a good tolerance.

Patients enrolled will be randomized in 2 groups:
Group 1: 30 patients will receive masitinib 3 mg/kg/day during 4 weeks then 4.5 mg/kg/day during 4 weeks and then 6 mg/kg/day (each switch being subjected to a toxicity control).
Group 2: 15 patients will receive placebo with the same administration plan as masitinib.

The following rule will be applied to define whether the dose of masitinib may be increase:
no severe suspected (or not assessable) adverse event was reported; and
no suspected (or not assessable) adverse event led to treatment interruption; and
no suspected (or not assessable adverse event) is ongoing at the time of the dose increase, regardless of its severity.

At the week-4 visit, if the patient did not present with a suspected or not assessable adverse event which was either severe, or leading to masitinib interruption, and if no suspected or not assessable adverse event is ongoing at week 4, regardless of its severity, the daily dose of masitinib will be increased to 4.5 mg/kg/day.

The patients presenting with non-severe suspected adverse event at the time of the dose increase can pursue the dose progression schedule with one-month delay.

At the week-8 visit, if the patient did not present with a suspected or not assessable adverse event which was either severe, or leading to masitinib interruption, and if no suspected or not assessable adverse event is ongoing at week 8, regardless of its severity, the daily dose of masitinib will be increased to 6 mg/kg/day.

Dose of study treatment according to patient's weight are indicated in the tables below:

TABLE 4
Dose of study treatment (mg) according
to patient's weight (3 mg/kg/day).

| | | 3 mg/kg/day | | |
|---|---|---|---|---|
| Patient's | | Daily | Morning* | Evening** |
| weight in kg | | dose (mg) | (mg) | (mg) |
| >40.9 | ≤55.5 | 100 | | 100 |
| >55.5 | 77.7 | 200 | 100 | 100 |
| >77.7 | 99.9 | 300 | 100 | 200 |
| >99.9 | | 400 | 200 | 200 |

*Morning: the tablets should be taken during breakfast. In case of nausea, the administration can take place during lunch.
**Evening: the tablets should be taken during dinner.

TABLE 5
Dose of study treatment (mg)
according to patient's weight (4.5 mg/kg/day).

| | | 4.5 mg/kg/day | | |
|---|---|---|---|---|
| Patient's | | Daily dose | Morning* | Evening** |
| weight in kg | | (mg) | (mg) | (mg) |
| >40.9 | ≤55.5 | 200 | 100 | 100 |
| >55.5 | 77.7 | 300 | 100 | 200 |
| >77.7 | 99.9 | 400 | 200 | 200 |
| >99.9 | | 500 | 200 | 200 + 100 |

TABLE 6
Dose of study treatment according to
patient's weight (6 mg/kg/day).

| | | 6 mg/kg/day | | |
|---|---|---|---|---|
| Patient's | | Daily dose | Morning* | Evening** |
| weight in kg | | (mg) | (mg) | (mg) |
| >40.9 | ≤55.5 | 300 | 100 | 200 |
| >55.5 | 77.7 | 400 | 200 | 200 |
| >77.7 | 99.9 | 500 | 200 | 200 + 100 |
| >99.9 | | 600 | 200 + 100 | 200 + 100 |

### MAIN INCLUSION CRITERIA

1. Men and women with idiopathic Parkinson's disease according to DSM IV criteria of more than 3 years' duration defined by the cardinal sign, Bradykinesia, plus the presence of at least 1 of the following: resting tremor, rigidity, or impairment of postural reflexes, and without any other known or suspected cause of Parkinsonism.

2. Cognitive impairment confirmed by Mini-Mental State Examination (MMSE) score >12 and <25.

3. Modified Hoehn and Yahr stage from 2 to 4.

4. Minimal duration of disease evolution of 2 years.

5. Patients treated for a minimum of 2 months with a stable dose of levodopa and/or memantine and/or amantadine and/or rivastigmine at baseline, with no changes foreseen in therapy throughout the study.

6. Patients with unilateral tremor at onset of the disease.

7. Patient with normal organ function defined as:
absolute neutrophils count (ANC)≥2.0×10⁹/L;
hemoglobin≥10 g/dL;
platelets (PTL)≥100×10⁹/L;
AST/ALT≤3×ULN;
bilirubin≤1.5×ULN;
creatinine clearance>60 mL/min;
albumin >1×LLN;
Proteinuria<30 mg/dL (1+) on the dipstick. If proteinuria is≥1+on the dipstick, 24-hour proteinuria must be<1.5 g/24 hours.

8. Male or female patient, aged≥40 years, weight≥41 kg and BMI between 18 and 35 kg/m².

### MAIN EXCLUSION CRITERIA

1. History of cardiac, hematologic, hepatic, renal, pancreatic, metabolic, respiratory, gastrointestinal, endocrinologic, or neurologic system condition or a tumor that is clinically significant for their participation in the study.

2. Patient with a diagnosis of PD Dementia (probable, possible) according to the Clinical Diagnostic Criteria for Dementia Associated with PD, active psychosis or hallucinations, severe depression or delirium.

3. Patient with a major surgery within 2 weeks prior to study entry.

### Mandatory Concomitant Treatments

Patients must have been treated for a minimum of 2 months with a stable dose of levodopa and/or memantine and/or amantadine and/or rivastigmine at baseline, with no changes in therapy throughout the study. Safety issues related to these treatments should be managed according to usual practice.

### CRITERIA FOR EVALUATION

### Primary Variable

Absolute change from baseline in ADCS-ADL score at week 48.

### Secondary criteria

Absolute change from baseline in ADAS-Cog score at week 48.

Absolute change from baseline in NPI-10 score at week 48.

Absolute change from baseline in CIBIC+score at week 48.

Absolute change from baseline in Mattis Dementia Rating Scale (DRS) score at week 48.

Absolute change from baseline in CDR scale score at week 48.

Absolute change from baseline in Mini-Mental State Examination (MMSE) score at week 48.

Absolute change from baseline in Modified Hoehn and Yahr stage at week 48.

Absolute change from baseline in UPDRS part I (mentation, behavior and mood) score at week 48.

Absolute change from baseline in UPDRS part II (Activity Daily Living) score at week 48.

Absolute change from baseline in UPDRS part III (motor) score at week 48.

Absolute change from baseline in Parkinson's Disease Questionnaire (PDQ-39) score at week 48.

### Analysis of primary endpoint—ADCS-ADL:

Only the global score (range from 0 to 78 where 78 implies full functioning with no impairment), which is the sum of the 23 items rated by the caregiver will be analyzed.

Comparison between treatment groups (masitinib arm versus placebo arm) will be performed on ADCS-ADL score absolute change from baseline at Week 48 by using a model of analysis of covariance with ADCS-ADL score baseline value as covariate and the following fixed factors:
Treatment;
Time onset of first symptoms.

Modified Intent-To-Treat (m-ITT) analysis will be used as primary population and ITT then Per Protocol as secondary populations. Modified Last Observation Carried Forward (mLOCF) method will be used as primary analysis for management of missing data. mLOCF is defined as follows:
For patients who discontinued early due to treatment-related reasons (AEs related or lack of efficacy), the LOCF method will be used.
For patients who discontinued early due to nontreatment-related reasons (i.e., others than AEs related or Lack of efficacy), Observed Cases will be used.
For patients who did not discontinue the study but had a missing data at Week 48, the LOCF method will be used.
For patients who died for related disease progression until W48:
   If patient was treated, PSPRS score is replaced by 100;
   If patient discontinued, and death occurred less than one month after discontinuation, PSPRS score is replaced by 100, else the LOCF method will be used.

### Sensitivity Analysis

1—Primary analysis will be repeated using Observed Cases.
2—Primary analysis will be repeated assuming LOCF as management of missing data.
3—Primary analysis will be repeated using a multiple Imputation method.
4—Same analysis as primary analysis and the sensitivity analysis 1), 2) and 3) will be done on ITT and PP populations.

These analyses should conclude in the same direction. If not, results will be discussed.

### Analysis of secondary endpoints:

ADAS-Cog score:

Comparison between treatment groups (masitinib-arm versus placebo-arm) will be performed on ADAS-Cog score absolute change from baseline at Week 48 by using a model of analysis of covariance. Baseline value (ADAS-Cog score) as covariate and the same factors as for the primary criterion analysis will be used in the model.
NPI-10:

Comparison between treatment groups (masitinib-arm versus placebo-arm) will be performed on NPI-10 score absolute change from baseline at Week 48 by using a model of analysis of covariance. Baseline value (NPI-10 score) as covariate and the same factors as for the primary criterion analysis will be used in the model.
CIBIC-plus:

Comparison between treatment groups (masitinib-arm versus placebo-arm) will be performed on the difference between improvements [1-3] and worsening [5-7] CIBIC-plus classes (improvement minus worsening) at Week 48 by using a model of analysis of covariance for repeated measures. To do so, CIBIC-plus will be considered as factor variable in the model and a specific contrast will be implemented to compare treatment groups (masitinib-arm versus placebo-arm) on the difference between worsening [5-7] and improvement [1-3] CIBIC-plus classes. Modified Intent To Treat (m-ITT) analysis will be used as primary population.
Mattis Dementia Rating Scale (DRS):

Comparison between treatment groups (masitinib-arm versus placebo-arm) will be performed on DRS score absolute change from baseline at Week 48 by using a model of analysis of covariance. Baseline value (DRS score) as covariate and the same factors as for the primary criterion analysis will be used in the model.
CDR:

Comparison between treatment groups (masitinib-arm versus placebo-arm) will be performed on CDR scale absolute change from baseline at Week 48 by using a model of analysis of covariance. Baseline values (CDR scale . . . ) as covariates and the same factors as for the primary criterion analysis will be used in the model.
Mini-Mental State Examination (MMSE):

Comparison between treatment groups (masitinib-arm versus placebo-arm) will be performed on MMSE score absolute change from baseline at Week 48 by using a model of analysis of covariance. Baseline value (MMSE score) as covariate and the same factors as for the primary criterion analysis will be used in the model.
Modified Hoehn and Yahr:

Comparison between treatment groups (masitinib-arm versus placebo-arm) will be performed on modified Hoehn and Yahr score absolute change from baseline at Week 48 by using a model of analysis of covariance. Baseline value (Modified Hoehn and Yahr score) as covariate and the same factors as for the primary criterion analysis will be used in the model.
UPDRS part I (mentation, behavior and mood):

Comparison between treatment groups (masitinib-arm versus placebo-arm) will be performed on UPDRS part I score absolute change from baseline at Week 48 by using a model of analysis of covariance. Baseline value (UPDRS part I score) as covariate and the same factors as for the primary criterion analysis will be used in the model.
UPDRS part II (Activity Daily Living):

Comparison between treatment groups (masitinib-arm versus placebo-arm) will be performed on UPDRS part II score absolute change from baseline at Week 48 by using a model of analysis of covariance. Baseline value (UPDRS part II score) as covariate and the same factors as for the primary criterion analysis will be used in the model.
UPDRS part III (motor):

Comparison between treatment groups (masitinib-arm versus placebo-arm) will be performed on UPDRS part III score absolute change from baseline to Week 12, Week 24, Week 36 and Week 48 by using a model of analysis of covariance. Baseline value (UPDRS part III score) as covariate and the same factors as for the primary criterion analysis will be used in the model.
Parkinson's Disease Questionnaire (PDQ-39):

Comparison between treatment groups (masitinib-arm versus placebo-arm) will be performed on PDQ-39 score absolute change from baseline at Week 48 by using a model of analysis of covariance. Baseline value (PDQ-39 score) as covariate and the same factors as for the primary criterion analysis will be used in the model.

1-19 (canceled)

20. A method for the treatment of Parkinson's disease in a mammal in need thereof, said method comprising administering to the mammal masitinib or a pharmaceutically acceptable salt or solvate thereof.

21. The method according to claim 20, wherein said mammal is a human patient.

22. The method according to claim 20, wherein the pharmaceutically acceptable salt of masitinib is a mesilate salt.

23. The method according to claim 20, wherein said masitinib, or a pharmaceutically acceptable salt or solvate thereof, is administered at a daily dose of 1.0 to 12.0 mg/kg (mg per kg bodyweight).

24. The method according to claim 20, wherein said masitinib, or a pharmaceutically acceptable salt or solvate thereof, is administered at a dose of 1.5, 3.0, 4.5, 6.0, 7.5, or 9.0 mg/kg/day (mg per kilo body weight per day).

25. The method according to claim 20, wherein said masitinib, or a pharmaceutically acceptable salt or solvate thereof, is administered at an initial dose of 3.0 mg/kg/day (mg per kilo body weight per day) during at least 4 weeks, then 4.5 mg/kg/day during at least 4 weeks, and at 6 mg/kg/day thereafter, with each dose escalation being subjected to toxicity controls.

26. The method according to claim 20, wherein said masitinib, or a pharmaceutically acceptable salt or solvate thereof, is administered in two daily intakes.

27. The method according to claim 20, wherein said masitinib, or a pharmaceutically acceptable salt or solvate thereof, is administered orally.

28. The method according to claim 20, wherein said masitinib, or a pharmaceutically acceptable salt or solvate thereof, is administered in combination with at least one other pharmaceutically active ingredient.

29. The method according to claim 28, wherein said at least one other pharmaceutically active ingredient is chosen from the group consisting of: levodopa, carbidopa-levodopa, dopamine agonists, monoamine oxidase B (MAO-B) inhibitors, catechol-O-methyl transferase (COMT) inhibitors, NMDA receptor antagonists, acetylcholinesterase inhibitors, and mixture thereof.

30. The method according to claim 28, wherein said at least one other pharmaceutically active ingredient is chosen from the group consisting of: levodopa, carbidopa-levodopa, bromocriptine, pergolide, pramipexole, ropinirole, piribedil, cabergoline, apomorphine, lisuride, safinamide, selegiline, rasagiline, entacapone, tolcapone, amantadine, memantine, rivastigmine, donepezil, galantamine, and mixture thereof.

31. The method according to claim 28, wherein said at least one other pharmaceutically active ingredient is chosen from the group consisting of: levodopa, memantine, amantadine, rivastigmine, and any mixture thereof.

32. The method according to claim 28, wherein said masitinib, or a pharmaceutically acceptable salt or solvate thereof, is administered in combination with said at least one other pharmaceutically active ingredient in a combined preparation for simultaneous, separate, or sequential use.
